(19) United States
(12) Patent Application Publication
(10) Pub. No.: US 2005/0175577 A1
(43) Pub. Date: Aug. 11, 2005

(54) Absorbent composition with improved odor control

(75) Inventors: Dennis B. Jenkins, Oakland, CA (US); Daniel E. Wheeler, Oakland, CA (US); Charles F. Fritter, Oakland, CA (US); Ananth N. Shenoy, Oakland, CA (US); David L. Deleeuw, Oakland, CA (US)

Correspondence Address: JOEL J. HAYASHIDA;CORPORATE PATENT COUNSEL, THE CLOROX COMPANY, P.O. BOX 24305, OAKLAND, CA, 94623-1305, US

(73) Assignee: THE CLOROX COMPANY

(21) Appl. No.: 10/773,585

(22) Filed: Feb. 6, 2004

(51) Int. Cl. A01K 29/00; A61L 9/01

(52) U.S. Cl. 424/76.1; 119/173

(57) ABSTRACT

An absorbent composition with improved odor control and suitable for use as an animal litter, comprising an absorbent material, activated alumina, and optional additives.

### BACKGROUND OF THE INVENTION

1. Field of the Invention

The invention relates to absorbent compositions suitable for use as an animal litter, and having activated alumina as an odor-inhibiting active.

2. Brief Statement of the Related Art

Because of the growing number of domestic animals used as house pets, there is a need for litters so that animals may micturate, void or otherwise eliminate liquid or solid waste indoors in a controlled location. However, inevitably, waste build-up leads to malodor production, which is offensive to the human olfactory senses.

The human objection to odor is not the only reason that it is desirable to reduce odors. Studies have shown that cats prefer litter with little or no animal smell. One theory is that cats like to mark their territory by urinating. When cats return to the litterbox and do not sense their odor, they will try to mark their territory again. The net effect is that cats return to use the litter box more often if the odor of their markings are reduced.

One solution to the malodor problem arising from used animal litter has been the introduction of a new form of cat litter comprising a litter composition which contains bentonite clay particles. Bentonite is a water-swellable clay which, upon contact with moist animal waste, is able to agglomerate with other moistened bentonite clay particles. This thus isolates the moist animal waste by the agglomeration of the moist clay particles. The agglomerations form an isolatable clump, which can be removed from the litter. Examples of this type of clumping or scoopable litter technology are disclosed in Hughes, U.S. Pat. Nos. 5,503,111; 5,386,803; 5,317,990; 5,129,365 and U.S. Reissue Patent RE 33,983, all of which are incorporated herein by reference.

On the other hand, boron-containing compounds, especially boric acid, have been identified as effective additives to both clumping and non-clumping clay-based animal litters. These are discussed in, for example, Ratcliffet al., U.S. Pat. Nos. 4,949,672, 5,094,190, and 5,992,351, Jenkins et al., U.S. Pat. No. 5,176,108, Stanislowski et al., U.S. Pat. Nos. 5,018,482, 5,135,743 and 5,183,655, all of which are incorporated herein by reference. Still other references have discussed the use of borax in a cat litter in which a water soluble polymer present is caused to gel or harden by the presence of borax as a reaction initiator or catalyst, but not as an odor control agent. See Goss et al., U.S. Pat. No. 5,359,961 and Richard, U.S. Pat. No. 5,183,010. Other patents discuss the use of borax, albeit in a non-clumping animal litter, for example, Clark et al., U.S. Pat. No. 3,352,792, and Christianson, U.S. Pat. No. 4,263,873.

Finally, Gordon, U.S. Pat. No. 4,641,605, discloses the use of various buffering agents, including sodium borate, in a litter in which a strong oxidant, sodium or ammonium persulfate is present to reduce odors in animal litters.

Activated alumina has long been known as a desiccant in gas-phase processes and applications. However, the art has been devoid of any teaching of the many beneficial properties of activated alumina in the context of animal litter.

Sawyer, U.S. Pat. No. 3,029,783 discloses the use of aluminum sulfate and aluminum chloride for controlling odors. These aluminum salts are formed by reacting an aluminiferous base material with sulfuric or hydrochloric acid.

Brewer, U.S. Pat. No. 3,921,581 uses raw alumina as a liquid-absorbing base material for a litter as well as a carrier for a fragrance.

However, none of the foregoing art teaches, discloses or suggests that activated alumina can reduce malodors in clumping and non-clumping litters. Further, none of the foregoing art discloses, teaches or suggests that this odor control—which is believed to be attributable to adsorption and absorption of odor-causing molecules—can be accomplished without hindering the adherence or agglomeration of clumpable clay litters when contacted with moisture.

Nor does the foregoing art teach, disclose or suggest the use of activated alumina in or as a liquid-absorbing composition useful for absorbing harmful and noxious chemicals such as spilled gasoline or motor oil.

### SUMMARY OF THE INVENTION

The invention provides an absorbent composition particularly useful as a clumping or nonclumping animal litter with improved odor control. In one embodiment, the absorbent composition includes optional absorbent material, optional additives, and to 100% activated alumina. In another embodiment, the absorbent composition includes a mixture of activated alumina and absorbent material, with optional additives. In yet another embodiment, the absorbent composition includes composite particles containing both activated alumina and absorbent material, with optional additives.

Significant odor control improvements over current commercial litter formulas have been identified for, but are not limited to, the following areas:
Fecal odor control (malodor source: feline feces)
Ammonia odor control (malodor source: feline urine)
Non-ammonia odor control (malodor source: feline urine)

The absorbent compositions described herein are useful for many types of uses other than as an animal litter. Such uses include, for example, filtration, bioremediation/hazardous/spill cleanup, pharma/ag applications, soaps, detergents, and other dry products, etc.

Other aspects and advantages of the present invention will become apparent from the following detailed description, which, when taken in conjunction with the drawings, illustrate by way of example the principles of the invention.

### BRIEF DESCRIPTION OF THE DRAWINGS

FIG. 1 illustrates several configurations of absorbent composite particles according to various embodiments of the present invention.

FIG. 2 is a process diagram illustrating a pan agglomeration process according to a preferred embodiment.

FIG. 3 depicts the structure of an illustrative agglomerated composite particle formed by the process of FIG. 2.

FIG. 4 is a process diagram illustrating another exemplary pan agglomeration process with a recycle subsystem.

FIG. 5 is a process diagram illustrating an exemplary pin mixer process for forming composite absorbent particles.

FIG. 6 is a process diagram illustrating an exemplary mix muller process for forming composite absorbent particles.

FIG. 7 is a graph illustrating odor control test results for several odor control agents.

### DETAILED DESCRIPTION OF THE PREFERRED EMBODIMENTS

The following description includes the best embodiments presently contemplated for carrying out the present invention. This description is made for the purpose of illustrating the general principles of the present invention and is not meant to limit the inventive concepts claimed herein.

The present invention relates generally to absorbent compositions with improved malodor controlling properties, the compositions comprising absorbent material, activated alumina, and optional performance-enhancing additives (actives). A preferred use for the compositions is as a cat litter, and therefore much of the discussion herein will refer to cat litter applications. However, it should be kept in mind that the absorbent compositions have a multitude of applications, and should not be limited to the context of a cat litter.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

Absorbent Materials

The absorbent material can be any material capable of absorbing a liquid such as animal urine. Many liquid-absorbing materials may be used without departing from the spirit and scope of the present invention. Illustrative absorbent materials include but are not limited to minerals, fly ash, absorbing pelletized materials, perlite, silicas, organics such as cellulosic materials, other absorbent materials and mixtures thereof. Preferred minerals include: bentonites, zeolites, fullers earth, attapulgite, montmorillonite diatomaceous earth, opaline silica, Georgia White clay, sepiolite, calcite, dolomite, slate, pumice, tobermite, marls, attapulgite, kaolinite, halloysite, smectite, vermiculite, hectorite, Fuller's earth, fossilized plant materials, expanded perlites, gypsum and other similar minerals and mixtures thereof.

The preferred absorbent material is sodium bentonite, also known as Wyoming bentonite. Bentonite clays are able to absorb many times their weight of a liquid and agglomerate with nearby wetted bentonite particles to form wet clumps which may be removed from a litterbox. The clay particles are typically comminuted. That is, they are pelletized, ground or formed into particles and screened to a size varying from about 0.05 to about 10,000 microns, although such particle size does not appear critical to the practice of the invention. A preferred particle size for bentonite clay particles is in the range of about 4700 microns to about 50 microns (˜4×200 U.S. mesh). A preferred bentonite particle size for clumping litter is in the range of about 3000 microns to about 100 microns (˜7×140 U.S. mesh), and ideally in the range of about 1400 microns to about 300 microns (˜14×50 U.S. mesh).

Bentonite fines having a size less than about 125 microns (100 U.S. mesh) may also be employed to produce some or all of the particles of absorbent material, and may exhibit both improved absorbency for feline urine and improved dry clump strength. Bentonite fines can be agglomerated through a process called “pin mixing” pursuant to which large amounts of water (up to 30% by weight based on the total weight of the bentonite) are added to the fines and the material is pin mixed under high shear and then dried, ground and sized.

Bentonite particles and fines can also be compacted to form particles, as described in U.S. Pat. No. 5,775,259 incorporated herein by reference. The compaction of water-swellable bentonite particles containing bentonite fines may be accomplished by a wide variety of compaction processes known in the art to effect size enlargement of small particles into larger particles. These larger particles are often referred to in the art as agglomerates, and the process of making the larger particles is often referred to as agglomeration. A particularly enlightening treatise on size enlargement by agglomeration is published by John Wiley & Sons, entitled “Size Enlargement by Agglomeration” by, Wolfgang Pietsch, (1991). A wide variety of presses may be used to provide the compacting pressures of this invention so as to form compacted water-swellable bentonite containing an effective amount of bentonite fines. One particularly useful process is the use of a press with rolls. This compaction process is generally referred to as “roll compaction” or “roll pressing”, since the material to be compacted is pressed between rollers rotating in opposite directions while applying pressure to continually advancing material. The aforementioned treatise discusses the process of roll compaction at pages 260 to 332, incorporated herein by reference thereto. In one embodiment, compaction is carried out by roll compaction by passing the water-swellable bentonite-containing material through opposing rollers urged together under a selected total pressure of at least 1000 pounds per square inch (gauge), preferably at least 1500 pounds per square inch (gauge) and, further, at a pressure of at least 3500 psig. Roll compaction pressures are often stated in terms of pounds per lineal inch (pli), and pressures of at least 5000 pli are believed suitable, with roll compaction pressures of at least 10,000 pli and more preferably at least 20,000 pli being useful herein. Roll compaction pressures of 28,000 pli have been found usable herein to form the compacted masses which contain effective amounts of bentonite fines. The surfaces of the rolls may be selected from a wide variety of surface textures and designs. The roll surfaces may be smooth or profiled so as to produce a continuous compacted bentonite, having a planar smooth shape, rod-shaped, briquette-shaped, corrugated shape, fluted shape or other selected shapes. After the water-swelled bentonite particles are compacted, the compacted bentonite mass is broken up by passing it through one or more grinding means selected to form a preselected particle size distribution, depending on selected absorbent use, from the compacted bentonite mass. The broken up bentonite mass from the grinding means is then passed through suitable sizing screens to give a final product having a preselected particle size range and/or particle size distribution. Compacted bentonite-containing particles which are too small or too large for the intended use can be recycled for compacting. Alternatively, particles too large for the intended use (e.g., animal litter) can be recycled by regrinding such bentonite particles and recycling the reground particles. Since the instant invention relates in its broadest sense to the compaction of water-swellable bentonite-containing particles containing bentonite fines the actual compaction means used for compacting the bentonite fines is more one of efficiency for commercial manufacturing as contrasted with being critical for obtaining the benefits observed. Among the numerous compacting processes and techniques known in the prior art which may be employed herein, include, but not limited to, pan agglomeration, roll compaction, roll briquetting, vertical hydraulic pressing, rotary tableting, gear pelleting and flat plate pelleting.

Activated Alumina

Activated alumina (Al₂O₃) has been found to provide odor control comparable or even superior to other odor control additives such as activated carbon, zeolites, and silica gel. Alumina is a white granular material, and is properly called aluminum oxide.

Typical aluminas include or are derived from gibbsite, boemite, pseudo boemite, and bauxite, each alumina potentially having different properties. The Bayer refining process used by alumina refineries worldwide involves four steps—digestion, clarification, precipitation and calcination. To turn bauxite into alumina, the ore is ground and mixed with lime and caustic soda. The mixture is pumped into high-pressure containers, and heated. The aluminum oxide is dissolved by the caustic soda, then precipitated out of this solution, washed, and heated to drive off water.

One process of making activated alumina includes a heating step, which dries and cracks the alumina particles to create fissures and pores that increase the absorptive ability of the alumina. The resulting product is a white, free flowing powder with a bulk density of about 40-60 lbs/ft³. A commercial supplier of activated alumina suitable for use in the embodiments presented herein is Alcoa, 201 Isabella Street, Pittsburgh, Pa. 15212-5858 USA. The preferred activated alumina material has been activated by a heat process, though chemical activation processes can also be used.

While not wishing to be bound by any particular theory, the inventors believe that the odor controlling properties of activated alumina are derived from a combination of adsorption and absorption. The porous and fissurous structure of the alumina provides a large surface area, and consequently, more sites for adsorption. Additionally, odiferous molecules may become physically trapped, or absorbed, in the pores and fissures of the alumina.

The particle size of the activated alumina used in the litter is not the largest contributor to the odor-controlling properties of the alumina,. However, the particle size of the alumina may be important to avoid segregation issues, namely that alumina having a particle size substantially smaller than the absorbent particles will tend to settle towards the bottom of the mixture. This settling may affect odor controlling properties of the alumina due to its physical location in the package (the amount of alumina in the mixture is not consistent) as well as in a litter box (the alumina should be generally homogenous throughout the mixture or located towards the top of the litter box where odors tend to escape to the atmosphere). Therefore, the preferred particle size of the activated alumina is selected such that it will not substantially segregate out of the mixture. This determination can be made on the basis of the particle size of alumina relative to the particle size of the absorbent material and additives, density of the materials relative to each other, etc. For example, where the absorbent material consists mainly of dried and crushed sodium bentonite particles in the particle size range of about 1.4 mm-0.3 mm (14×50 mesh), the activated alumina particles are preferably in the range of about 1-2 mm (10×18 mesh).

Because the smaller particle size may improve odor controlling properties of activated alumina, powdered activated alumina can be coated onto the particles of absorbent material. Also, the activated alumina can be formed into composite particles with one or more absorbent materials and optional additives. A description of such composite particles is provided below.

Particles of activated alumina in an effective amount can be dry mixed with the other components of the absorbent composition. Preferably, the activated alumina is present in the composition in an amount of about 0.01% to about 50% of the composition by weight based on the total weight of the absorbent composition. More preferably, the activated alumina is present in the composition in an amount of about 0.1% to about 25% by weight.

Absorbent compositions can also be formed from 100% activated alumina. Other compositions can be formed primarily of activated alumina (e.g., >80-90%) with other additives and absorbent materials.

Additives

Illustrative additives include but are not limited to antimicrobials, odor absorbers/inhibitors, binders, dedusting agents, fragrances, health indicating materials, nonstick release agents, superabsorbent materials, lightweight materials, colorants, and mixtures thereof.

Preferred antimicrobial actives are boron containing compounds such as borax pentahydrate, borax decahydrate, boric acid, polyborate, tetraboric acid, sodium metaborate, anhydrous borate, boron components of polymers, and mixtures thereof. The antimicrobial active can be added as a solid and dry mixed into the mixture, or can be sprayed onto the particles in the mixture. Antimicrobial actives are preferably added in an amount of up to about 1%. More information about the effects of boron-containing compounds in cat litter is found in U.S. Pat. No. 5,992,351, which is herein incorporated by reference.

Odor control actives that supplement the alumina may also be added. One type of odor absorbing/inhibiting active inhibits the formation of odors. An illustrative material is a water soluble metal salt such as silver, copper, zinc, iron, and aluminum salts and mixtures thereof. Preferred metallic salts are zinc chloride, zinc gluconate, zinc lactate, zinc maleate, zinc salicylate, zinc sulfate, zinc ricinoleate, copper chloride, copper gluconate, and mixtures thereof. Other odor control actives include metal oxide nanoparticles. Additional types of odor absorbing/inhibiting actives include cyclodextrin, zeolites, activated carbon, acidic, salt-forming materials, and mixtures thereof.

Some antimicrobial actives also provide an odor-controlling benefit. For example, borax, or, more accurately, di-alkali metal tetraborate n—hydrate (preferably, Na₂B₄O₇×nH₂O, where n=4, 5 or 10), appears to provide multiple benefits in odor control by: (1) acting as a urease inhibitor, which controls odors by preventing enzymatic breakdown of urea; (2) having bacteriostatic properties, which appear to help control odor by controlling the growth of bacteria which are responsible for production of the urease enzymes.

Nonstick release agents such as calcium bentonite or baking soda can be added to reduce and potentially eliminate sticking to a litter box.

The additive may also include a clumping aid or binder such as lignin sulfonate (solid), polymeric binders, fibrillated Teflon® (polytetrafluoroethylene or PTFE), and combinations thereof. Useful organic polymerizable binders include, but are not limited to, carboxymethylcellulose (CMC) and its derivatives and its metal salts, guar gum cellulose, xanthan gum, starch, lignin, polyvinyl alcohol, polyacrylic acid, styrene butadiene resins (SBR), and polystyrene acrylic acid resins. Water stable composite particles can also be made with crosslinked polyester network, including but not limited to those resulting from the reactions of polyacrylic acid or citric acid with different polyols such as glycerin, polyvinyl alcohol, lignin, and hydroxyethylcellulose.

The natural tendency of bentonite and other inorganic clays is to form dust upon handling as a result of attrition of the particles during handling and shipping. Dedusting agents such as colloidal polytetrafluoroethylene can be added to the particles in order to reduce the dust ratio. Many of the binders listed above are also effective dedusting agents when applied to the outer surface of the absorbent particles. Other dedusting compounds or agents include but are not limited to gums, water-soluble polymeric resins, e.g., polyvinyl alcohol, polyvinyl acetate, polyvinyl pyrrolidone, polyacrylic acid, xanthan gum, gum arabic, other natural resins and mixtures of any of these resins.

A color altering agent such as a dye, pigment, bleach, lightener, etc. may be added to vary the color of particles, such as to lighten the overall color of the litter so it is more appealing to an animal, aid a consumer in distinguishing the alumina from the other materials, etc. For instance, suitable dyes include, but are not limited to, direct dyes, vat dyes, sulfur dyes, acid dyes, mordant acid dyes, premetalized acid dyes, basic dyes, dispersed dyes, reactive dyes, azo dyes, phthalocyanine dyes, anthraquinone dye, quinoline dyes, monoazo, diazo and polyazo dyes, and suitably treated titanium dioxide. Preferred dyes include anthraquinone, quinoline and monoazo dyes. Especially preferred dyes are polymeric dyes (e.g., dyes that are covalently bonded to polymers). Illustrative pigments include phthalo pigments. Other types of color altering agents include non-staining coloring agents, especially of the type that do not stain the material to which applied until dried.

The activated alumina itself may include an embedded coloring agent that has been added during the fabrication of the activated alumina particles. The inventors have found that the odor absorbing properties of activated alumina are not significantly reduced due to the application of color altering agents thereto.

Additionally, activated alumina's natural white coloring makes it a desirable choice as a white, painted or dyed “speckle” in litters. In composite and other particles, the activated alumina can also be added in an amount sufficient to lighten or otherwise alter the overall color of the particle or the overall color of the entire composition.

Compositions may also contain visible but ineffective colored speckles for visual appeal. Examples of speckle material are salt crystals or gypsum crystals.

Preferably, the color altering agent comprises up to approximately 5% of the absorbent composition, more preferably, 0.001%-1% of the composition. Even more preferably, the color altering agent comprises approximately 0.001%-0.01% of the composition.

In a further aspect of the invention, the color altering agent is disposed on one or more of the materials such that at least 10% of the overall absorbent composition is colored. More preferably, the colorant agent is disposed on at least 20% of the materials. Zeolite, alumina and silica gel are preferred carriers for the color altering agent. Zeolite is preferred, as it has a density similar to that of bentonite, the preferred primary absorbent material, and so will not tend to significantly migrate during packaging, transport, or use.

According to the invention, the color altering agents may be any color, even yellow. An effective amount of dye or pigment is that which is perceived by consumers to be preferred over uncolored litter. One well established method of assessing the effectiveness of the dye or pigment is by measuring the litter composition resistance to color changes in the b region (or coordinate) of the L,a,b color scale when soiled by animal urine. As is well known in the art, the L,a,b color scale is a uniform color system developed by Hunterlab to represent colors. See, e.g., Kirk-Othmer, Encyclopedia of Chemical Technology, 4^{th} Ed., Vol. 11, p. 238 (1994); R. S. Hunter, Instruments and Test Methods for Control of Whiteness in Textile Mills, Proceedings of the American Association of Textile Chemists and Colorists, 1966 National Technical Conference (1966).

Fragrances (such as those available from such commercial vendors as Quest, Sozio, Bush Boake and Allen, Firmenich, Mane U.S.A., International Flavours and Fragrances, Inc., Dragoco, Noville, Belmay and Givaudan) are optionally added. Such fragrances can additionally be uncoated (e.g., fragrance blends) or encapsulated (as in U.S. Pat. No. 4,407,231). Fragrance can be added in an amount up to about 10%, preferably up to about 5%, and ideally in an amount less than about 1%. Fragrances can include those that are aesthetically appealing to a human or that mask odor. Other fragrances include animal attractants.

Animal health indicating actives may also be added to the composition, or packages separately for addition to the mixture in the litter box. One such active includes a pH indicator that changes color when urinated upon, thereby indicating a health issue with the animal. U.S. Pat. No. 6,308,658, incorporated by reference, describes a litmus agent that visually indicates the presence of a urinary infection in animals. Another type of active detects and indicates occult blood in animal urine.

Because minerals, and particularly clay, are heavy, it is may be desirable to reduce the weight of the composite absorbent particles to reduce shipping costs, reduce the amount of material needed to need to fill the same relative volume of the litter box, and to make the material easier for customers to carry. Exemplary lightweight materials that may be added to the composition include but are not limited to calcium bentonite clay, attapulgite clay, perlite, silica, zeolite, non-absorbent silicious materials, sand, plant seeds, glass, polymeric materials, wood pulp and other cellulosics, and mixtures thereof. As an example, the preferred absorbent material is sodium bentonite, which has a density of about 70 lbs/ft³. By adding a lighter material such silica (25 lbs/ft³) or zeolite (about 50 lbs/ft³), the overall weight per volume unit of the mixture can be reduced.

Suitable superabsorbent materials include superabsorbent polymers such as AN905SH, FA920SH, and F04490SH, all from Floerger. Preferably, the superabsorbent material can absorb at least 5 times its weight of water, and ideally more than 10 times its weight of water.

TABLE 1

| ADDITIVE | QUANTITY (wt %) |
|---|---|
| Metal Perborates or Metal Borates | 0.01 wt % to 20 wt % |
| Dyes-urine activated color dyes | 1 ppm to 12,000 ppm |
| Citric Acid and salts of citric acid | 0.1 wt % to 5 wt % |
| Dye/Metal Perborates or Metal Borates | 0.1 wt % to 5 wt % |
| | (ratio of 1:5 to 1:50) |
| Starch | 0.5 wt % to 5.0 wt %; |
| | Preferred 2.0 wt % to 4.0 wt % |
| Guar Gum | 0.5 wt % to 2.0 wt %; |
| | Preferred 1.0 wt % to 1.5 wt % |
| Sodium Bicarbonate or | 0.5 wt % to 10.0 wt %; |
| Potassium Bicarbonate | Preferred 2.0 wt % to 5.0 wt % |
| Citric Acid or salts of citric acid | 0.5 wt % to 10.0 wt %; |
| | Preferred 2.0 wt % to 5.0 wt % |
| Water-Dispersible Dye | 1 ppm to 12,000 ppm; |
| FD & C Blue No. 1 | Preferred 6,000 ppm to |
| (Brilliant Blue FCF) | 10,000 ppm |
| FD & C Green No. 3 (Fast Green FCF) | |
| Activated Carbon or other | .01 wt % to 10 wt %; |
| carbonaceous absorbent | Preferred 1.0 wt % to 3.0 wt % |
| Zeolites and/or other molecular | .01 wt % to 10 wt %; |
| sieves | Preferred 1.0 wt % to 3.0 wt % |
| Spray-Dried Fragrance | ˜50% loading; |
| | 0.01 wt % to 10 wt %; |
| | 250 ppm to 1000 ppm |
| | Oil on a carrier (starch beads) |

ppm = parts per million

Composite Particles

The present invention also includes compositions that incorporate composite particles containing absorbent material and optionally performance-enhancing actives (activated alumina and/or other additives). For example, the composite particles can be formed of the absorbent material alone, absorbent material+alumina, absorbent material+additives, and absorbent material+alumina+additives. The absorbent compositions can include combinations of any of these particles, and can also include particles of alumina and/or additives dry mixed with the composite particles.

The composite absorbent particles have improved physical and chemical properties. By using the processes and materials described in copending U.S. patent application Ser. No. 10/618,401, filed Jul. 11, 2003, which is herein incorporated by reference, as well as activated alumina as an active, such particles can be “engineered” to preferentially exhibit specific characteristics including but not limited to improved odor control, lower density, easier scooping, better particle/active consistency, higher clump strength, etc. One of the many benefits of this technology is that the alumina and/or other performance-enhancing actives may be positioned to optimally react with target molecules such as but not limited to odor causing volatile substances, resulting in surprising odor control with very low levels of active ingredient. One great advantage of the particles of the present invention is that substantially every absorbent particle can be made to contain activated alumina.

One or more performance-enhancing actives (additives) are preferably added to the particles in an amount effective to perform the desired functionality or provide the desired benefit. For example, these actives can be added during the agglomeration process so that the actives are incorporated into the particle itself, or can be added during a later processing step. Illustrative materials for the performance-enhancing active(s) include but are not limited to activated alumina, antimicrobials, odor absorbers/inhibitors, binders, fragrances, health indicating materials, nonstick release agents, superabsorbent materials, and mixtures thereof.

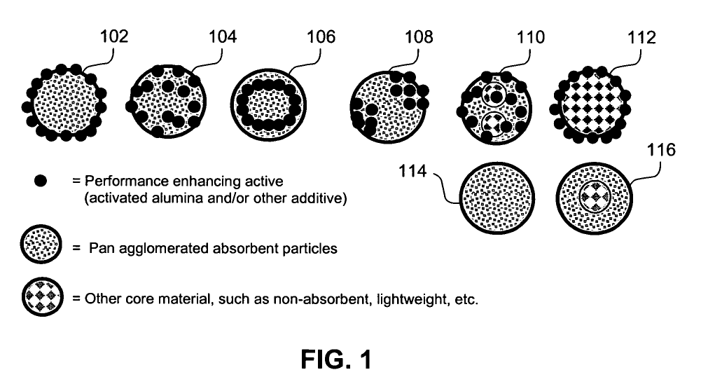

FIG. 1 shows several embodiments of the absorbent particles of the present invention. These particles have actives (activated alumina and/or other actives) incorporated:
1. In a layer on the surface of a particle (102)
2. Evenly (homogeneously) throughout a composite litter particle (104)
3. In a concentric layer(s) throughout the particle and/or around a core (106)
4. In pockets or pores in and/or around a particle (108)
5. In a particle with single or multiple cores (110)
6. Utilizing non-absorbent cores (112)
7. No actives (114)
8. No actives, but with single or multiple cores (116)
9. In any combination of the above

A preferred embodiment is to bind activated alumina and/or other actives directly to the surface of composite absorbent particles. The use of actives bound only to the surface of absorbent particles leads to the following benefits:
1. the use of extremely small particle size of the active material results in a very high surface area of active while using a very small amount of active,
2. with actives present only on the surface of the substrate, the waste of expensive actives that would be found with ‘homogeneous’ composite particles [where actives are found throughout the substrate particles] is eliminated,
3. segregation of actives from substrates is eliminated; thus, the actives remain dispersed and do not end up on the bottom of the litter container,
4. by reducing the amount of expensive actives, the cost of the product is greatly reduced,
5. binding of small particle size actives directly to the substrate surface results in lower dust levels than in bulk added product.

Surprisingly, activated alumina has been found to provide excellent odor control in cat litter when they are bound to the surface of a material such as sodium bentonite clay. For example, binding of small amounts of activated alumina particles to sodium bentonite substrate particles using xanthan gum or fibrillatable PTFE as binder results in litter materials with superior odor adsorbing performance. In this example, the activated alumina is highly effective at capturing malodorous volatile organic compounds as they escape from solid and liquid wastes due to the high surface area of the activated alumina, and its preferred location on the surface of the sodium bentonite particles.

Another aspect of the invention is the use of encapsulated actives, where the actives are positioned inside the particle, homogeneously and/or in layers. Because of the porous structure of the particles, even actives positioned towards the center of the particle are available to provide their particular functionality. Encapsulation of actives provides a slow release mechanism such that the actives are in a useful form for a longer period of time. This is particularly so where the active is used to reduce malodors.

Generally, the preferred mean particle diameter of the activated alumina particles used to form composite particles is less than about 500 microns, but can be larger. A more preferred particle size of the activated is about 150 microns (˜100 mesh U.S.S.S.) or less, and ideally in the range of about 25 to 150 microns, with a mean diameter of about 50 microns (˜325 mesh U.S.S.S.) or less.

The composite particles can be dry mixed with other types of particles, including but not limited to other types of composite particles, extruded particles, particles formed by crushing a source material, etc. Mixing composite particles with other types of particles provides the benefits provided by the composite particles while allowing use of lower cost materials, such as crushed or extruded bentonite. Illustrative ratios of composite particles to other particles can be 75/25, 50/50, 25/75, or any other ratio desired. For example, in an animal litter created by mixing composite particles with extruded bentonite, a ratio of 50/50 will provide enhanced odor control, clumping and reduced sticking, while reducing the weight of the litter and lowering the overall cost of manufacturing the litter.

The composite particles can also be dry mixed with actives, including but not limited to particles of activated alumina and additives bound to carriers.

One preferred method of forming the absorbent particles is by agglomerating granules of an absorbent material in a pan agglomerator. A preferred pan agglomeration process is set forth in more detail below, but is described generally here to aid the reader. Generally, the granules of absorbent material are added to an angled, rotating pan. A fluid or binder is added to the granules in the pan to cause binding of the granules. As the pan rotates, the granules combine or agglomerate to form particles. Depending on pan angle and pan speed among other factors, the particles tumble out of the agglomerator when they reach a certain size. The particles are then dried and collected.

The agglomeration process in combination with the unique materials used allows the manufacturer to control the physical properties of particles, such as bulk density, dust, strength, as well as PSD (particle size distribution) without changing the fundamental composition and properties of absorbent particles.

One benefit of the pan agglomeration process of the present invention is targeted active delivery, i.e., the position of the active can be “targeted” to specific areas in, on, and/or throughout the particles. Another benefit is that because the way the absorbent particles are formed is controllable, additional benefits can be “engineered” into the absorbent particles, as set forth in more detail below.

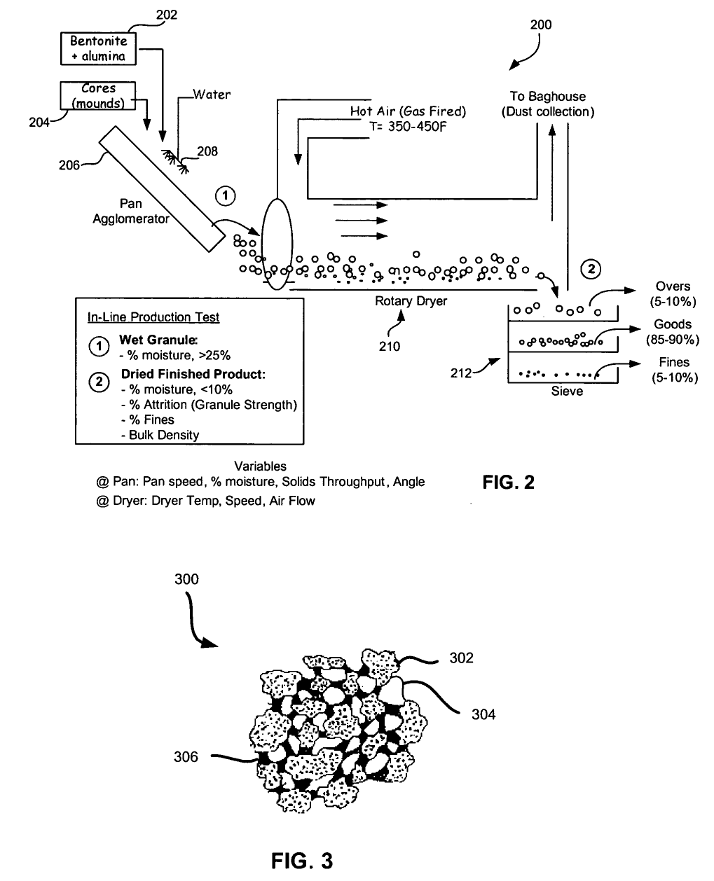

FIG. 2 is a process diagram illustrating a pan agglomeration process 200 according to a preferred embodiment. In this example, the absorbent granules are bentonite clay and the active is activated alumina. Cores of a suitable material, here calcium bentonite clay, are also added. The absorbent particles (e.g., bentonite powder) is mixed with the active (e.g., activated alumina) to form a dry mixture, which is stored in a hopper 202 from which the mixture is fed into the agglomerator 206. Alternatively, the absorbent granules and active(s) may be fed to the agglomerator individually. For example, liquid actives can be added by a sprayer. The cores are preferably stored in another hopper 204, from which they are fed into the agglomerator. A feed curtain can be used to feed the various materials to the agglomerator.

In this example, the agglomerator is a pan agglomerator. The pan agglomerator rotates at a set or variable speed about an axis that is angled from the vertical. Water and/or binder is sprayed onto the granules in the agglomerator via sprayers 208 to raise/maintain the moisture content of the particles at a desired level so that they stick together. Bentonite acts as its own binder when wetted, causing it to clump, and so additional binder is not be necessary. The pan agglomeration process gently forms composite particles through a snowballing effect broadly classified by experts as natural or tumble growth agglomeration. FIG. 3 depicts the structure of an illustrative agglomerated composite particle 300 formed during the process of FIG. 2. As shown, the particle includes granules of absorbent material 302 and active 304 with moisture 306 or binder positioned interstitially between the granules.

Depending on the pan angle and pan speed, the particles tumble off upon reaching a certain size. Thus, the pan angle and speed controls how big the particles get. The particles are captured as they tumble from the agglomerator. The particles are then dried to a desired moisture level by any suitable mechanism, such as a rotary or fluid bed. In this example, a forced air rotary dryer 210 is used to lower the high moisture content of the particles to less than about 15% by weight and ideally about 8-13% by weight. At the outlet of the rotary dryer, the particles are screened with sieves 212 or other suitable mechanism to separate out the particles of the desired size range. Tests have shown that about 80% or more of the particles produced by pan agglomeration will be in the desired particle size range. Preferably, the yield of particles in the desired size range is 85% or above, and ideally 90% or higher. The selected particle size range can be in the range of about 10 mm to about 100 microns, and preferably about 2.5 mm or less. An illustrative desired particle size range is 12×40 mesh (1650-400 microns).

The exhaust from the dryer is sent to a baghouse for dust collection. Additional actives such as borax and fragrance can be added to the particles at any point in the process before, during and/or after agglomeration. Also, additional/different actives can be dry blended with the particles.

Illustrative composite absorbent particles after drying have a specific weight of from about 0.15 to about 1.2 kilograms per liter and a liquid absorbing capability of from about 0.6 to about 2.5 liters of water per kilogram of particles. Preferably, the particles absorb about 50% or more of their weight in moisture, more preferably about 75% or more of their weight in moisture, even more preferably greater than approximately 80% and ideally about 90% or more of their weight in moisture.

The following table lists illustrative properties for various compositions of particles created by a 20″ pan agglomerator at pan angles of 40-60 degrees and pan speeds of 20-50 RPM. The total solids flow rates into the pan were 0.2-1.0 kg/min.

TABLE 2

| | | Bentonite | | Bulk | |
|---|---|---|---|---|---|
| | | to | Final | Density | Clump |
| Core | Water | Core Ratio | Moisture | (kg/l) | Strength |
| None | 15-23% | 100:0 | 1.0-1.4% | 0.70-0.78 | 95-97 |
| Calcium | 15-23 | 50:50 | 3.4 | 0.60-0.66 | 95-97 |
| bentonite | | | | | |
| Calcium | 15-18 | 33:67 | 4.3-4.4 | 0.57-0.60 | 93-95 |
| bentonite | | | | | |
| Sand | 10-12 | 50:50 | 2.0 | 0.81-0.85 | 97-98 |
| Sand | 6-8 | 33:67 | 1.6-2.4 | 0.92 | 97 |
| Perlite | 15-19% | 84:16 | | 0.36-0.39 | 97% |
| Perlite | 16-23% | 76:24 | | 0.27-0.28 | 95-97% |

Clump strength is measured by first generating a clump by pouring 10 ml of pooled cat urine (from several cats so it is not cat specific) onto a 2 inch thick layer of litter. The urine causes the litter to clump. The clump is then placed on a ½″ screen after a predetermined amount of time (e.g., 6 hours) has passed since the particles were wetted. The screen is agitated for 5 seconds with the arm up using a Ro-Tap Mechanical Sieve Shaker made by W. S. Tyler, Inc. The percentage of particles retained in the clump is calculated by dividing the weigh of the clump after agitation by the weight of the clump before agitation. Referring again to the table above, note that the clump strength indicates the percentage of particles retained in the clump after 6 hours. As shown, >90%, and more ideally, >95% of the particles are retained in a clump after 6 hours upon addition of an aqueous solution, such as deionized water or animal urine. Note that ≧about 80% particle retention in the clump is preferred. Also, note the reduction in bulk density when a core of calcium bentonite clay or perlite is used.

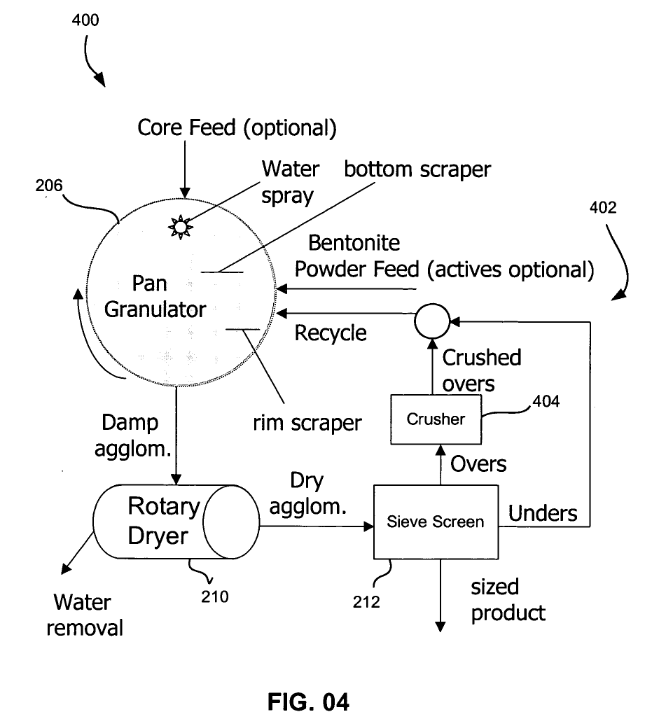

FIG. 4 is a process diagram illustrating another exemplary pan agglomeration process 400 with a recycle subsystem 402. Save for the recycle subsystem, the system of FIG. 4 functions substantially the same as described above with respect to FIG. 2. As shown in FIG. 4, particles under the desired size are sent back to the agglomerator. Particles over the desired size are crushed in a crusher 404 and returned to the agglomerator.

The diverse types of clays and mediums that can be utilized to create absorbent particles should not be limited to those cited above. Further, unit operations used to develop these particles include but should not be limited to: high shear agglomeration processes, low shear agglomeration processes, high pressure agglomeration processes, low pressure agglomeration processes, mix mullers, roll press compacters, pin mixers, batch tumble blending mixers (with or without liquid addition), and rotary drum agglomerators. For simplicity, however, the larger portion of this description shall refer to the pan agglomeration process, it being understood that other processes could potentially be utilized with similar results.

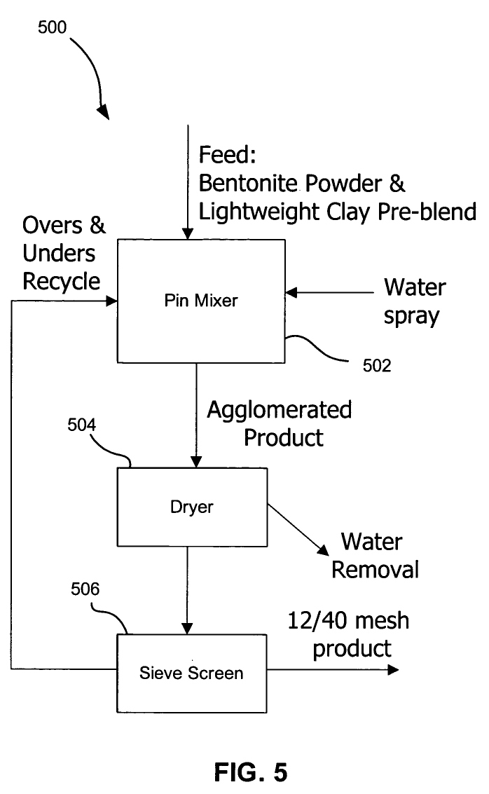

FIG. 5 is a process diagram illustrating an exemplary pin mixer process 500 for forming composite absorbent particles. As shown, absorbent particles and active are fed to a pin mixer 502. Water is also sprayed into the mixer. The agglomerated particles are then dried in a dryer 504 and sorted by size in a sieve screen system 506. The following table lists illustrative properties for various compositions of particles created by pin mixing.

TABLE 3

| | Bentonite | | | |
|---|---|---|---|---|
| | to Clay | Water | Bulk | Clump Strength |
| Lightweight | Ratio | Addition | Density | −6 hours |
| Clay | (wt %) | (wt %) | (lb/ft³) | (% Retained) |
| Zeolite | 50:50 | 20 | 59 | 91 |
| Bentonite | 100:0 | 20 | 67 | 95 |

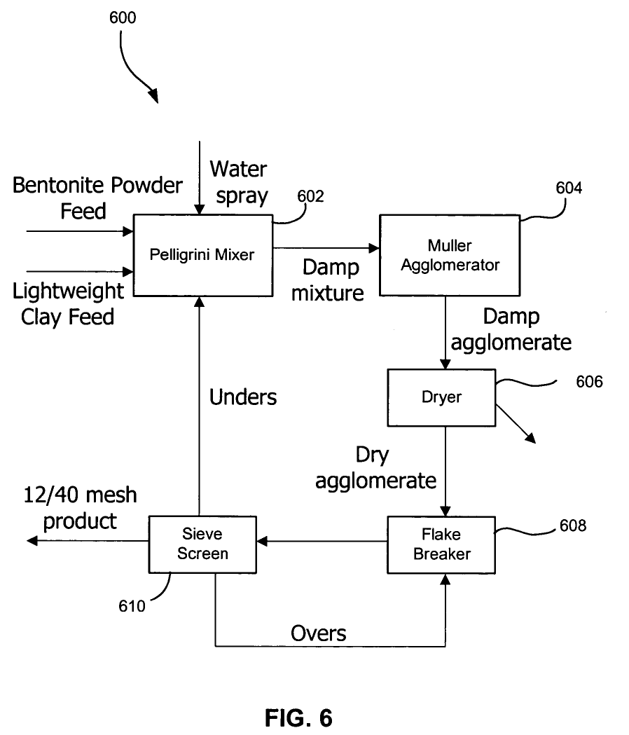

FIG. 6 is a process diagram illustrating an exemplary mix muller process 600 for forming composite absorbent particles. As shown, the various components and water and/or binder are added to a pellegrini mixer 602. The damp mixture is sent to a muller agglomerator 604 where the mixture is agglomerated. The agglomerated particles are dried in a dryer 606, processed in a flake breaker 608, and then sorted by size in a sieve screen system 610. The following table lists illustrative properties for various compositions of particles created by a muller process. Note that the moisture content of samples after drying is 2-6 weight percent.

TABLE 4

| | | | | | Clump | |
|---|---|---|---|---|---|---|
| | | | Calcu- | | Strength - | |
| | | | lated | Actual | 6 | |
| | Bentonite: | Water | Bulk | Bulk | hours | |
| | Clay | Addition | Density | Density | (% | Dust |
| Clay | (wt %) | (wt %) | (lb/ft³) | (lb/ft³) | Retained) | (mg) |
| GWC* | 50:50 | 33 | 43 | 45 | 83 | 39 |
| GWC* | 50:50 | 47 | 43 | 42 | 56 | 34 |
| Taft DE** | 50:50 | 29 | 33 | 46 | 86 | 38 |
| Taft DE** | 50:50 | 41 | 33 | 43 | 76 | 35 |

*Georgia White Clay
**Taft Diatomaceous Earth

Other particle-forming processes may be used to form the composite particles of the present invention. For example, without limitation, extrusion and fluid bed processes appear appropriate. Extrusion process typically involves introducing a solid and a liquid to form a paste or doughy mass, then forcing through a die plate or other sizing means. Because the forcing of a mass through a die can adiabatically produce heat, a cooling jacket or other means of temperature regulation may be necessary. The chemical engineering literature has many examples of extrusion techniques, equipment and materials, such as “Outline of Particle Technology,” pp. 1-6 (1999), “Know-How in Extrusion of Plastics (Clays) or NonPlastics (Ceramic Oxides) Raw Materials, pp. 1-2, “Putting Crossflow Filtration to the Test,” Chemical Engineering, pp. 1-5 (2002), and Brodbeck et al., U.S. Pat. No. 5,269,962, especially col. 18, lines 30-61 thereof, all of which is incorporated herein by reference thereto. Fluid bed process is depicted in Coyne et al., U.S. Pat. No. 5,093,021, especially col. 8, line 65 to col. 9, line 40, incorporated herein by reference.

The composite absorbent particle can be formed into any desired shape. For example, the particles are substantially spherical in shape when they leave the agglomeration pan. At this point, i.e., prior to drying, the particles may have a high enough moisture content that they are malleable. By molding, compaction, or other processes known in the art, the composite absorbent particle (as well as any of the particles described herein) can be made into spheres and non-spherical shapes such as, for example, ovals, flattened spheres, hexagons, triangles, squares, etc. and combinations thereof.

### EXAMPLES

The following nonlimiting examples illustrate both general and specific implementations. Unless otherwise noted, the percentage of each element is by weight based on the total weight of the absorbent composition. Also note that any moisture content is presumed included in the various materials unless otherwise noted.

### Example 1

An absorbent composition (clumpable or nonclumpable) with improved odor control includes:
about 0.1-25.0% activated alumina particles
about 0-75% additives
to 100% particles of absorbent material

### Example 2

An absorbent composition with antimicrobial benefit includes:
about 0.5-5.0% activated alumina particles [odor control]
about 0.001-1.0% borax pentahydrate [antimicrobial]
about 0.001-10% fragrance
about 0-25% additional additives
to 100% swellable sodium bentonite clay particles

### Example 3

A clumping absorbent composition with antimicrobial benefit includes:
about 2% colored activated alumina particles, 1-2 mm (10×18 mesh)
about 0.5% borax pentahydrate [antimicrobial]
about 0.71% spray-dried fragrance—sprayed onto starch beads and mixed in
about 96.79% swellable sodium bentonite clay particles, ˜1.4 mm-0.3 mm (14×50 mesh), dried and crushed

### Example 4

As mentioned above, because the activated alumina particles typically have a different bulk density than the particles of absorbent material, segregation of the activated alumina can occur. The following composition provides the benefit of improved odor control throughout the litter due to the varying densities of zeolite, activated, alumina, and silica gel.

An absorbent composition that is either clumpable or nonclumpable includes:
about 0.001-25.0% zeolite particles
about 0.001-25.0% activated alumina particles
about 0.001-25.0% silica gel particles
about 0-50% additives
to 100% particles of absorbent material

The zeolite is the heaviest of the three odor-absorbing materials, alumina is in the middle, and silica gel is the lightest. Because of the tendency of the materials to segregate upon agitation such as a cat digging in the litterbox, the zeolite, being heavier, will tend to move towards the bottom of the litter, while the lighter silica gel will tend to migrate towards the top of the litter. Thus, the litter will contain odor controlling actives throughout. An additional benefit is that the silica gel tends to repel liquid running across it, making it the ideal material for the upper layer of litter, as it will not immediately become saturated by animal urine but will retain its odor absorbing properties.

Also, by adding a lighter material such silica (25 lbs/ft³) or zeolite (about 50 lbs/ft³), the overall weight per volume unit of the mixture is reduced.

For clumping litter not relying on binders for clump strength, the total content of zeolite, activated alumina, and silica gel particles is preferably less than about 25% so that the clay provides satisfactory clumping performance.

### Example 5

In a variation of Example 4:

An absorbent composition that is either clumpable or nonclumpable includes:
about 0.001-25.0% activated alumina particles
about 0.001-25.0% zeolite particles
about 0-50% additives
to 100% particles of absorbent material

### Example 6

In a variation of Example 4:

An absorbent composition that is either clumpable or nonclumpable includes:
about 0.001-25.0% activated alumina particles
about 0.001-25.0% silica gel particles
about 0-50% additives
to 100% particles of absorbent material

### Example 7

An absorbent composition:
about 0-50% additives
to 100% activated alumina particles

### Example 8

A flushable and clumping absorbent composition with improved odor control includes:
about 0.1-25.0% activated alumina particles
about 0-75% additives
less than about 1% of a water soluble binding agent
to 100% particles of absorbent material

The following Examples describe several composite particles:

### Example 9

Referring again to FIG. 1, a method for making particles 102 is generally performed using a pan agglomeration process in which clay particles of ≦200 mesh (≦74 microns), preferably ≦325 mesh (≦43 microns) particle size premixed with particles of activated alumina, are agglomerated in the presence of an aqueous solution to form particles in the size range of about 12×40 mesh (about 1650-250 microns). Alternatively, the particles are first formed with clay alone, then reintroduced into the pan or tumbler, and the activated alumina is added to the pan or tumbler, and a batch run is performed in the presence of water or a binder to adhere the activated alumina to the surface of the particles. Additional actives can be premixed with the clay, added to the agglomeration pan, added to the composite particles after agglomeration, sprayed onto the composite particles during or after agglomeration, etc.

### Example 10

A method for making particles 104 is generally performed using the process described with relation to FIG. 2, except no core material is added.

### Example 11

A method for making particles 106 is generally performed using the process described with relation to FIG. 2, except that introduction of the absorbent granules and the active into the agglomerator are alternated to form layers of each.

### Example 12

A method for making particles 108 is generally performed using the process described with relation to FIG. 2, except that the activated alumina (and/or other active) has been pre-clumped using a binder, and the clumps are added. Alternatively, particles of absorbent material can be created by agglomeration and spotted with a binder such that upon tumbling with the activated alumina and/or another active, the activated alumina/active sticks to the spots of binder thereby forming concentrated areas. Yet another alternative includes the process of pressing clumps of activated alumina and/or active into the absorptive material.

### Example 13

A method for making particles 110 is generally performed using the process described with relation to FIG. 2.

### Example 14

A method for making particles 112 is generally performed using the process described with relation to FIG. 2.

### Example 15 & 16

A method for making particles 114 and 116 are generally performed using the process described with relation to FIG. 2, except no activated alumina or other active is added to the composite particle. Such particles can then be dry-mixed with activated alumina.

### Example 17

In addition, the performance-enhancing active can be physically dispersed along pores of the particle by suspending an insoluble active in a slurry and spraying the slurry onto the particles. The suspension travels into the pores and discontinuities, depositing the active therein.

Testing

Gas chromatography testing was performed on raw activated alumina to compare its odor controlling properties relative to other odor controlling substances. During the test, glass beads are placed in a glass vial. Particles of activated alumina are placed above the glass beads. A long needle having a mixture of target molecules is added to the vial below the glass beads so that any absorption is a gaseous absorption as opposed to a liquid absorption. In these experiments, the target molecules are esters, alcohols, and acids, which simulate odiferous molecules generated by animal waste. The vial is capped for about 24 hours to allow equilibration to occur. The vial is placed in a gas chromatography apparatus. A probe of the chromatography apparatus enters the vial to analyze the headspace in the vial, providing a count representing the amount of target molecules remaining.

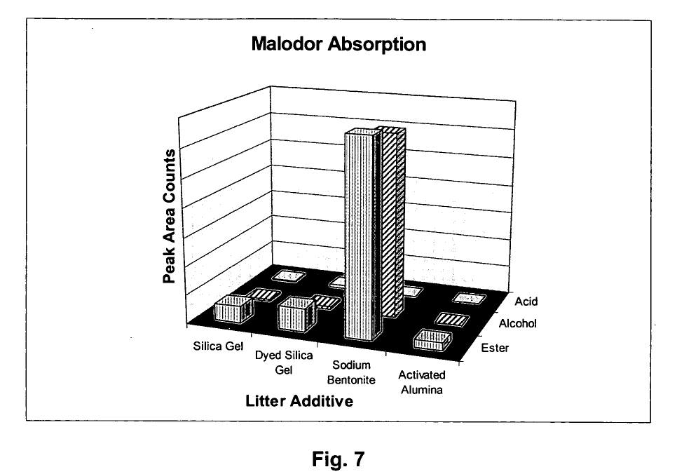

The table below illustrates the results of testing for several odor control agents. FIG. 7 graphically illustrates the results. As shown, activated alumina provides superior adsorption (lower odor) as compared to other odor control agents.

### TABLE 5

| Litter Additive | Esters | Alcohols | Acids |
|---|---|---|---|
| Silica Gel | Better | Better | Good |
| Dyed Silica Gel | Better | Better | Good |
| Sodium Bentonite | Good | Good | Best |
| Activated Alumina | Best | Best | Best |

It should be noted that the compositions of the present invention can be used in litter boxes or in cages of a wide variety of animals including common pets, cats, dogs, gerbils, guinea pigs, mice and hamsters, rabbits, ferrets and laboratory animals (e.g., mice, rats, and the like). The animal litter of the present invention is especially useful for smaller household animals, such as cats.

The compositions described above can be used as a “clumping” animal litter to selectively remove liquid animal wastes from a weight of animal litter by: contacting the animal litter with liquid animal waste thereby producing an agglomerated mass (generally referred to as a “clump”) comprising the animal litter and the liquid animal waste that is of sufficient size and of sufficient clumping strength to be removed from the litter and a remaining amount of litter; and removing the clump from the remaining amount of litter. Although the clump can be removed as a wet clump, owing to the use patterns of cat owners the clump is generally removed after it has dried at room temperature for a period of about 24 hours, thereby effectively removing the liquid animal waste from the remaining amount of litter. Owing to the moisture on the surface of solid animal wastes, the instant litters are also effective in adhering to solid animal wastes. In addition, the animal litter can be used with litter boxes of known designs. Such litter boxes are water-impermeable receptacles having disposed therein a litter comprising a compacted bentonite according to this invention and capable of agglomerating upon wetting into a clump of sufficient size and of sufficient clump strength for physical removal of the clump from the litter box. The removal of the clump is without substantial adherence to an animal, when either a wet clump or dry clump form.

As mentioned above, the compositions described herein have particular application for use as an animal litter. However, the particles should not be limited to pet litters, but rather could be applied to a number of other applications such as:
Litter Additives—Formulated product can be pre-blended with standard clumping or non-clumping clays to create a less expensive product with some of the benefits described herein. A post-additive product could also be sprinkled over or as an amendment to the litter box.
Filters—Air or water filters could be improved by either optimizing the position of activated alumina and actives into areas of likely contact, such as the outer perimeter of a filter particle. Composite particles with each subcomponent adding a benefit could also be used to create multi-functional composites that work to eliminate a wider range of contaminants.
Bioremediation/Hazardous/Spill Cleanup—The absorbent compositions described herein are useful for absorbing spilled liquid such as oil spills. Absorbents with actives specifically chosen to attack a particular waste material can also be engineered using the technology described herein. Exemplary waste materials include toxic waste, organic waste, hazardous waste, and non-toxic waste.

Pharma/Ag—Medications, skin patches, fertilizers, herbicides, insecticides, all typically use carriers blended with actives. Utilization of the technology described herein reduce the amount of active used (and the cost) while increasing efficacy.

Soaps, Detergents, and other Dry Products—Most dry household products could be engineered to be lighter, stronger, longer lasting, or cheaper using the technology as discussed herein.

Mixtures of Different Particles—The particles can be dry mixed with other types of particles, including but not limited to other types of composite particles, extruded particles, particles formed by crushing a source material, etc. Mixing various types of particles provides the desired benefits while allowing use of lower cost materials, such as crushed or extruded bentonite. Where composite particles are used, illustrative ratios of composite particles to other particles can be 75/25, 50/50, 25/75, or any other ratio desired. For example, in an animal litter created by mixing composite particles with extruded bentonite, a ratio of 50/50 will provide enhanced odor control, clumping and reduced sticking, while reducing the weight of the litter and lowering the overall cost of manufacturing the litter.
Mixtures of Composite Particles with Actives—The composite particles can be dry mixed with actives, including but not limited to particles of activated carbon.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

1. An absorbent composition, comprising:
particles of an absorbent material; and
particles of activated alumina dry mixed with the particles of absorbent material, the activated alumina being present in an amount of about 0.01% to about 50% by weight based on a total weight of the composition.

2. An absorbent composition as recited in claim 1, wherein the absorbent material is selected from a group consisting of: a mineral, fly ash, absorbing pelletized material, perlite, silica, organic materials, and mixtures thereof.

3. An absorbent composition as recited in claim 1, wherein the absorbent material is a mineral selected from a group consisting of: bentonite, zeolite, montmorillonite, diatomaceous earth, opaline silica, Georgia White clay, sepiolite, calcite, dolomite, slate, pumice, tobermite, marls, attapulgite, kaolinite, halloysite, smectite, vermiculite, hectorite, Fuller's earth, fossilized plant materials, expanded perlite, gypsum, and mixtures thereof.

4. An absorbent composition as recited in claim 1, wherein the particles of absorbent material have a particle size in a range from about 0.05 to about 10,000 microns.

5. An absorbent composition as recited in claim 1, further comprising a performance-enhancing active selected from a group consisting of: an antimicrobial, an odor reducing material, a binder, a fragrance, an animal health indicating material, a color altering agent, a dust reducing agent, a nonstick release agent, a superabsorbent material, cyclodextrin, zeolite, activated carbon, a pH altering agent, a salt forming material, a transition metal salt, and mixtures thereof.

6. An absorbent composition as recited in claim 1, further comprising a color altering agent selected from a group consisting of dye, pigment, bleach, lightener, non-staining coloring agent, embedded coloring agent, and mixtures thereof.

7. An absorbent composition as recited in claim 1, wherein the activated alumina is present in an amount of about 0.1% to about 15% by weight based on a total weight of the composition.

8. An absorbent composition as recited in claim 1, wherein the activated alumina is present in an amount of about 15% to about 50% by weight based on a total weight of the composition.

9. An absorbent composition as recited in claim 1, wherein the activated alumina is present in an amount of less than about 5% by weight based on a total weight of the composition.

10. An absorbent composition as recited in claim 1, wherein the particles of activated alumina have a particle size in a range from about 0.05 to about 10,000 microns.

11. An absorbent composition as recited in claim 1, wherein the particles of activated alumina have a particle size in a range from about 1,000 to about 2,000 microns.

12. An absorbent composition as recited in claim 1, wherein a particle size of the activated alumina is selected based on a particle size and density of the absorbent material such that segregation of the activated alumina in the composition is minimized.

13. An absorbent composition as recited in claim 1, wherein colorant is added to the particles of activated alumina.

14. An absorbent composition as recited in claim 1, wherein activated alumina is coated onto the particles of absorbent material.

15. An absorbent composition as recited in claim 1, wherein particles of activated alumina and absorbent material are combined in composite particles.

16. An absorbent composition as recited in claim 1, further comprising particles of a material selected from a group consisting of zeolite and silica gel.

17. An absorbent composition as recited in claim 1, further comprising baking soda for reducing sticking of the composition to a container upon wetting.

18. An absorbent composition as recited in claim 1, wherein the composition is capable of clumping upon wetting.

19. An absorbent composition as recited in claim 1, wherein the composition is flushable.

20. An absorbent composition as recited in claim 1, wherein the composition is capable of clumping upon wetting and is flushable.

21. An animal litter with improved odor control, comprising:
0 to about 50% of at least one additive; and
up to 100% activated alumina by weight based on a total weight of the composition.

22. An animal litter as recited in claim 21, further comprising water-swellable clay particles capable of adhering to other such particles upon contact with moisture.

23. An animal litter as recited in claim 21, wherein the additive is selected from a group consisting of: a mineral, fly ash, absorbing pelletized material, perlite, silica, organic materials, and mixtures thereof.

24. An animal litter as recited in claim 21, wherein the additive is a mineral selected from a group consisting of: bentonite, zeolite, montmorillonite, diatomaceous earth, opaline silica, Georgia White clay, sepiolite, calcite, dolomite, slate, pumice, tobermite, marls, attapulgite, kaolinite, halloysite, smectite, vermiculite, hectorite, Fuller's earth, fossilized plant materials, expanded perlite, gypsum, and mixtures thereof.

25. An animal litter as recited in claim 21, wherein the particles of additive have a particle size distribution in a range from about 0.05 to about 10,000 microns.

26. An animal litter as recited in claim 21, further comprising a performance-enhancing active selected from a group consisting of: an antimicrobial, an odor reducing material, a binder, a fragrance, an animal health indicating material, a color altering agent, a dust reducing agent, a nonstick release agent, a superabsorbent material, cyclodextrin, zeolite, activated carbon, a pH altering agent, a salt forming material, a transition metal salt, and mixtures thereof.

27. An animal litter as recited in claim 21, further comprising a color altering agent selected from a group consisting of dye, pigment, bleach, lightener, non-staining coloring agent, embedded coloring agent, and mixtures thereof.

28. An animal litter as recited in claim 21, wherein the activated alumina is present in an amount of about 0.1% to about 15% by weight based on a total weight of the composition.

29. An animal litter as recited in claim 21, wherein the activated alumina is present in an amount of less than about 5% by weight based on a total weight of the composition.

30. An animal litter as recited in claim 21, wherein a particle size of the activated alumina is selected based on a particle size and density of the absorbent material such that segregation of the activated alumina in the composition is minimized.

31. An animal litter as recited in claim 21, wherein colorant is added to the particles of activated alumina.

32. An animal litter as recited in claim 21, wherein activated alumina is coated onto the particles of absorbent material.

33. An animal litter as recited in claim 21, wherein particles of activated alumina and absorbent material are combined in composite particles.

34. An animal litter as recited in claim 21, further comprising particles of a material selected from a group consisting of zeolite and silica gel.

35. An animal litter as recited in claim 21, further comprising baking soda for reducing sticking of the composition to a container upon wetting.

36. An animal litter as recited in claim 21, wherein the composition is flushable.

37. An absorbent composition, comprising:
0 to about 50% of at least one additive; and
up to 100% activated alumina by weight based on a total weight of the composition.

38. An absorbent composition as recited in claim 37, wherein the additive is selected from a group consisting of: an antimicrobial, an odor reducing material, a binder, a fragrance, an animal health indicating material, a color altering agent, a dust reducing agent, a nonstick release agent, a superabsorbent material, cyclodextrin, zeolite, activated carbon, a pH altering agent, a salt forming material, a transition metal salt, and mixtures thereof.

39. An absorbent composition as recited in claim 37, wherein the particles of activated alumina have a particle size in a range from about 0.05 to about 10,000 microns.

40. A composite particle, comprising:
an absorbent material formed into a particle; and
activated alumina added to the absorbent material.

41. A composite particle as recited in claim 40, wherein the absorbent material is a liquid-absorbing material and is selected from a group consisting of: a mineral, fly ash, absorbing pelletized material, perlite, silica, organic materials, and mixtures thereof.

42. A composite particle as recited in claim 40, wherein the absorbent material is a mineral selected from a group consisting of: bentonite, zeolite, montmorillonite, diatomaceous earth, opaline silica, Georgia White clay, sepiolite, calcite, dolomite, slate, pumice, tobermite, marls, attapulgite, kaolinite, halloysite, smectite, vermiculite, hectorite, Fuller's earth, fossilized plant materials, expanded perlite, gypsum, and mixtures thereof.

43. A composite particle as recited in claim 40, wherein the absorbent material comprises sodium bentonite granules having a mean particle diameter of about 5000 microns or less.

44. A composite particle as recited in claim 40, wherein the absorbent material comprises sodium bentonite granules having a mean particle diameter of about 3000 microns or less.

45. A composite particle as recited in claim 40, wherein the absorbent material comprises sodium bentonite granules having a mean particle diameter in the range of about 25 to about 150 microns.

46. A composite particle as recited in claim 40, further comprising a performance-enhancing active includes at least one of an antimicrobial, an odor reducing material, a binder, a fragrance, a health indicating material, a color altering agent, a dust reducing agent, a nonstick release agent, a superabsorbent material, cyclodextrin, zeolite, activated carbon, a pH altering agent, a salt forming material, a transition metal salt and mixtures thereof.

47. A composite particle as recited in claim 40, further comprising a color altering agent selected from a group consisting of dye, pigment, bleach, lightener, non-staining coloring agent, embedded coloring agent, and mixtures thereof.

48. A composite particle as recited in claim 40, wherein the activated alumina is added in an amount sufficient to lighten an overall color of the composite particle as compared to a particle containing identical materials except the activated alumina.

49. A composite particle as recited in claim 40, wherein the activated alumina is sprayed onto the particles.

50. A composite particle as recited in claim 40, wherein granules of activated alumina are dry-blended with the particles.

51. A composite particle as recited in claim 40, wherein the activated alumina is present in an effective amount to control odors.

52. A composite particle as recited in claim 40, wherein the activated alumina is present in about 5 weight percent or less based on a weight of the composite particle.

53. A composite particle as recited in claim 40, wherein the activated alumina is present in about 1 weight percent or less based on a weight of the composite particle.

54. A composite particle as recited in claim 40, wherein the activated alumina has a mean particle diameter of about 5000 microns or less.

55. A composite particle as recited in claim 40, wherein the activated alumina has a mean particle diameter of about 1500 microns or less.

56. A composite particle as recited in claim 55, wherein the activated alumina has a mean particle diameter of about 50 microns or less.

57. A composite particle as recited in claim 40, wherein the activated alumina is substantially homogeneously dispersed throughout at least a portion of the absorbent particle.

58. A composite particle as recited in claim 40, wherein the activated alumina is physically dispersed in at least one layer.

59. A composite particle as recited in claim 40, wherein the activated alumina is physically dispersed in pockets in the particle.

60. A composite particle as recited in claim 40, wherein the activated alumina is physically dispersed in at least one position selected from along surfaces of the particle and contained within pores of the particle.

61. A composite particle as recited in claim 40, further comprising an absorbent core, the absorbent material being coupled to the core.

62. A composite particle as recited in claim 40, further comprising a non-absorbent core, the absorbent material being coupled to the core.

63. A composite particle as recited in claim 40, further comprising a hollow core, the absorbent material being coupled to the core.

64. A composite particle as recited in claim 40, further comprising a core, the absorbent material at least partially surrounding the core in the form of a shell, wherein an average thickness of the shell is at least about four times an average diameter of the core.

65. A composite particle as recited in claim 40, further comprising a core, the absorbent material at least partially surrounding the core in the form of a shell, wherein an average thickness of the shell is between about 1 and about 4 times an average diameter of the core.

66. A composite particle as recited in claim 40, further comprising a core, the absorbent material at least partially surrounding the core in the form of a shell, wherein an average thickness of the shell is less than an average diameter of the core.

67. A composite particle as recited in claim 40, further comprising a core, the absorbent material at least partially surrounding the core in the form of a shell, wherein an average thickness of the shell is less than about one-half an average diameter of the core.

68. A composite particle as recited in claim 40, further comprising a heavy core comprised of a material having a density higher than a density of the absorbent material, the absorbent material being coupled to the core.

69. A composite particle as recited in claim 40, further comprising a lightweight core comprised of a material having a density lower than a density of the absorbent material, the absorbent material being coupled to the core.

70. A composite particle as recited in claim 40, further comprising a core comprised of a pH-altering material, the absorbent material being coupled to the core.

71. A composite particle as recited in claim 40, wherein the particle has a bulk density of less than about 90% of a bulk density of a generally solid particle containing the absorbent material alone.

72. A composite particle as recited in claim 40, wherein the particle has a bulk density of less than about 70% of a bulk density of a generally solid particle containing the absorbent material alone.

73. A composite particle as recited in claim 40, wherein the particle has a bulk density of less than about 50% of a bulk density of a generally solid particle containing the absorbent material alone.

74. A composite particle as recited in claim 40, further comprising multiple cores, the absorbent material being coupled to the cores.

75. A composite particle as recited in claim 40, wherein the composite particle has a hydraulic conductivity value of about 0.25 cm/s or less.

76. A composite particle as recited in claim 40, wherein the composite particle exhibits reduced sticking to a container in which the composite particle rests when the particle is wetted relative to a generally solid particle under substantially similar conditions.

77. A composite particle as recited in claim 40, wherein the composite particle has a moisture content of less than about 25% by weight based on a weight of the composite particle.

78. A composite particle as recited in claim 40, wherein the composite particle has a moisture content of less than about 15% by weight based on a weight of the composite particle.

79. A composite particle as recited in claim 40, wherein the composite particle has a moisture content of less than about 10% by weight based on a weight of the composite particle.

80. A composite particle as recited in claim 40, wherein the composite particle is capable of absorbing a weight of water equaling at least about 90 percent of a weight of the composite particle.

81. A composite particle as recited in claim 40, wherein the composite particle is capable of absorbing a weight of water equaling at least about 75 percent of a weight of the composite particle.

82. A composite particle as recited in claim 40, wherein the composite particle is capable of absorbing a weight of water equaling at least about 50 percent of a weight of the composite particle.

83. A composite particle as recited in claim 40, wherein the composite particle has a dusting attrition value of at most about 15% as measured by ASTM method E-728 Standard Test Method for Resistance to Attrition of Granular Carriers and Granular Pesticides.

84. A composite particle as recited in claim 40, wherein the composite particle exhibits noticeably less odor after four days from contamination with animal waste as compared to a generally solid particle of the absorbent material alone under substantially similar conditions.

85. A composite particle as recited in claim 40, wherein the composite particle has been formed by an agglomeration process.

86. A composite particle as recited in claim 85, wherein the agglomeration process is a pan agglomeration process.

87. A composite particle as recited in claim 85, wherein the agglomeration process is at least one of a high shear agglomeration process, a low shear agglomeration process, a high pressure agglomeration process, a low pressure agglomeration process, a rotary drum agglomeration process, a fluid bed agglomeration process, a mix muller process, a roll press compaction process, a pin mixer process, a batch tumble blending mixer process, an extrusion process and a fluid bed process.

88. A composite particle as recited in claim 40, wherein the composite particle has a bulk density of about 1.5 grams per cubic centimeter or less.

89. A composite particle as recited in claim 40, wherein the composite particle has a bulk density of 0.85 grams per cubic centimeter or less.

90. A composite particle as recited in claim 89, wherein the composite particle has a bulk density of between about 0.25 and 0.85 grams per cubic centimeter.

91. A composite particle as recited in claim 40, wherein the particle has a liquid absorbing capability of from about 0.6 to about 2.5 liters of water per kilogram of particles.

92. A composite particle as recited in claim 40, wherein the particle is used in at least one of an animal litter product, a laundry product, a home care product, a water filtration product, an air filtration product, a fertilizer product, an iron ore pelletizing product, a pharmaceutical product, an agricultural product, a waste and landfill remediation product, a bioremediation product, and an insecticide product.

93. A composite particle as recited in claim 40, wherein substantially each particle includes activated alumina.

94. A composite particle as recited in claim 40, wherein substantially each particle includes activated alumina and at least one other additive.

95. Multiple composite particles as recited in claim 40, wherein some of the particles include a first active, and other particles contain a second active, the second active being different than the first active.

96. Multiple composite particles as recited in claim 40, wherein at least about 80% of the particles are retained in a clump upon addition of an aqueous solution.

97. Multiple composite particles as recited in claim 40, wherein at least about 90% of the particles are retained in a clump upon addition of an aqueous solution.

98. Multiple composite particles as recited in claim 40, wherein at least about 95% of the particles are retained in a clump after 6 hours upon addition of 10 ml of cat urine.

99. A litterbox with an absorbent composition disposed therein, comprising:
a receptacle with a closed bottom and a plurality of interconnected generally upright side walls forming an open top;
particles of an absorbent material disposed in the receptacle; and
particles of activated alumina disposed in the receptacle.

100. An absorbent composition, comprising:
0 to about 50% of at least one additive; and
to 100% of an aluminum-containing material by weight based on a total weight of the composition, wherein the aluminum containing material is derived from at least one of gibbsite, boemite, pseudo boemite, and bauxite.

101. An absorbent composition, comprising:
particles of an absorbent material; and
secondary particles selected from a group consisting of activated alumina and zeolite, the secondary particles being dry mixed with the particles of absorbent material, the secondary particles being present in an amount of about 0.01% to about 50% by weight based on a total weight of the composition.

102. An absorbent composition as recited in claim 1, wherein the absorbent material is selected from a group consisting of: a mineral, fly ash, absorbing pelletized material, perlite, silica, organic materials, and mixtures thereof.

103. An absorbent composition as recited in claim 1, wherein the absorbent material is a mineral selected from a group consisting of: bentonite, zeolite, montmorillonite, diatomaceous earth, opaline silica, Georgia White clay, sepiolite, calcite, dolomite, slate, pumice, tobermite, marls, attapulgite, kaolinite, halloysite, smectite, vermiculite, hectorite, Fuller's earth, fossilized plant materials, expanded perlite, gypsum, and mixtures thereof.

104. An absorbent composition as recited in claim 1, further comprising a color altering agent applied to the at least one of the activated alumina and the zeolite, the color altering agent being selected from a group consisting of dye, pigment, bleach, lightener, non-staining coloring agent, embedded coloring agent, and mixtures thereof.

105. An absorbent composition, comprising:
particles of an absorbent material; and
colored particles mixed with the particles of absorbent material, the colored particles with a bulk density of about from 50% to 150% that of the absorbent material.

106. An absorbent composition as recited in claim 105 wherein the bulk density of the colored material is about from 70% to 130% that of the absorbent material.

107. An absorbent composition as recited in claim 105 wherein the bulk density of the colored material is about from 90% to 110% that of the absorbent material.

108. An absorbent composition as recited in claim 105 wherein the bulk density of the colored material is about equivalent to that of the absorbent material.

109. An absorbent composition as recited in claim 105 wherein the colored material is zeolite.

110. An absorbent composition as recited in claim 105, wherein the absorbent material is a mineral selected from a group consisting of: bentonite, montmorillonite, diatomaceous earth, opaline silica, Georgia White clay, sepiolite, calcite, dolomite, slate, pumice, tobermite, marls, attapulgite, kaolinite, halloysite, smectite, vermiculite, hectorite, Fuller's earth, fossilized plant materials, expanded perlite, gypsum, and mixtures thereof.

111. An absorbent composition as recited in claim 105, wherein a color altering agent is used to make the colored particle, the color altering agent being selected from a group consisting of dye, pigment, bleach, lightener, non-staining coloring agent, embedded coloring agent, and mixtures thereof.

112. An absorbent composition as recited in claim 105, wherein the absorbent composition is an animal litter

113. A composite particle, comprising:
an absorbent material formed into a particle; and
zeolite added to the absorbent material.
